(19) United States
(12) Patent Application Publication
(10) Pub. No.: US 2008/0194982 A1
(43) Pub. Date: Aug. 14, 2008

(54) Method and Apparatus for Inductively Measuring the Bio-Impedance of a Users Body

(75) Inventors: Gerd Lanfermann, Aachen (DE); Jeroen Adrianus Johannes Thijs, Aachen (DE); Robert Pinter, Aachen (DE); Claudia Hannelore Igney, Karlsruhe (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 11/917,526

(22) PCT Filed: Jun. 20, 2006
(86) PCT No.: PCT/IB2006/051981

§ 371 Date: Dec. 14, 2007

(30) Foreign Application Priority Data
Jun. 23, 2005 (EP) 05105604.2

(51) Int. Cl. A61B 5/053

(52) U.S. Cl. 600/547

(57) ABSTRACT

In order to provide a simple and reliable method and apparatus for determining the position and/or the motion of a user's body during inductively measuring the bio-impedance of that body an apparatus (1) is suggested, which apparatus (1) comprises generating means (3) adapted to induce an alternating magnetic field in the user's body (2), the apparatus further comprising a number of reference signal generators (4) attached to the user's body (2), each reference signal generator (4) being adapted to generate a reference signal, the apparatus further comprising sensing means (6) adapted to measure a secondary magnetic field to obtain bio-impedance values and further adapted to measure a number of reference signals, and the apparatus further comprising analyzing means (8) adapted to determine the position and/or the motion of the user's body (2) based on the measured reference signals.

The present invention relates to a method and apparatus for inductively measuring the bio-impedance of a user's body.

The inductive measurement of bio-impedance is a known method to determine various vital parameters and images of the conductivity distribution of a human body in a non-contact way. The operating principle is the following: Using an inductor loop, an alternating magnetic field is induced in a part of the human body. This alternating magnetic field causes eddy currents in the tissue of the body. Depending on the type and conductivity of tissue, the eddy currents are stronger or weaker. The eddy currents cause a secondary magnetic field, which can be measured as an induced voltage in a second inductor loop (measurement coil).

The inductive measurement of the bio-impedance has been shown to allow the non-contact determination of several parameters, e.g. breath action and depth, heart rate and change of the heart volume and blood glucose level, as well as fat or water content of the tissue.

Bio-impedance measurements can be used in a tomographic way to obtain a conductivity distribution over time in a user's body in two or three dimensions or as a single image. From measurements over time, certain characteristics, like volume changes of the heart or lungs of the user can be calculated. However, movements of the user during measurement cause motion artifact. Then it is for example unclear, if the area of interest is moving due to its own dynamics or because the user turns around, e.g. if the user is staying in bed.

It is known to determine the position of a user during an impedance scan by applying a visual approach using laser beams and cameras. A profile of the body is provided and body movements during the impedance scan are monitored. A disadvantage of this solution is, that it is difficult to use in the context of personal healthcare for an unobtrusive monitoring at night. Furthermore it requires much more effort in addition to the impedance scanning. Finally it is not practical for long term measurements, e.g. while the user is sleeping.

Another solution is suggested by U.S. Pat. No. 6,445,943, which describes a position tracking and imaging system for use in medical applications. This system is characterized in that the position of a medical instrument is monitored with respect to a patient's body and for displaying at least one of a plurality of prerecorded images of the body responsive to the position of the medical instrument. For the purpose of position tracking a reference unit is applied to the patient. Furthermore a field generator is provided to generate an electromagnetic field. A field sensor senses the strength of the electromagnetic field. A position detecting unit is in communication with the field sensor and generates position data representing the position of the medical instrument with respect to the reference unit. A disadvantage of this solution is the large number of components necessary to track the position of the patient. Another disadvantage is the fact that reference signals that move relative to each other can cause false measurements. In U.S. Pat. No. 6,445,943 the medical instrument as a reference can move and distort the measurement.

It is an object of the present invention to provide a simple and reliable method and apparatus for determining the position and/or the motion of a user's body sequentially to and/or in parallel with inductively measuring the bio-impedance of that body.

This object is achieved according to the invention by an apparatus for inductively measuring the bio-impedance of a user's body, the apparatus comprising generating means which are adapted to induce an alternating magnetic field in the user's body, the apparatus further comprising a number of reference signal generators attached to the user's body, each reference signal generator being adapted to generate a reference signal, the apparatus further comprising sensing means which are adapted to measure a secondary magnetic field to obtain bio-impedance values and which are further adapted to measure a number of reference signals, and the apparatus further comprising analyzing means adapted to determine the position and/or the motion of the user's body based on the measured reference signals.

This object is also achieved by a method for inductively measuring the bio-impedance of a user's body, said method comprising the steps of attaching a number of reference signal generators to the user's body, operating the reference signal generators such that a number of reference signals is generated, inducing an alternating magnetic field in the user's body by generating means, measuring a secondary magnetic field by sensing means in order to obtain bio-impedance values, measuring a number of reference signals by said sensing means, and determining the position and/or the motion of the user's body based on the measured reference signals by analyzing means.

This object is also achieved by a computer program for use in a method for inductively measuring the bio-impedance of a user's body, said method comprising the steps of attaching a number of reference signal generators to the user's body, operating the reference signal generators such that a number of reference signals is actively generated, inducing an alternating magnetic field in the user's body by generating means, measuring a secondary magnetic field by sensing means in order to obtain bio-impedance values, and measuring a number of reference signals by said sensing means,
said computer program comprising computer instructions to determine the position and/or the motion of the user's body based on the measured reference signals, when the computer program is executed in a computer.

The technical effects necessary according to the invention can thus be realized on the basis of the instructions of the computer program in accordance with the invention. Such a computer program can be stored on a carrier such as a CD-ROM or it can be available over the internet or another computer network. Prior to execution, the computer program is loaded into the computer by reading the computer program from the carrier, for example by means of a CD-ROM player, or from the internet, and storing it in the memory of the computer. The computer includes inter alia a central processor unit (CPU), a bus system, memory means, e.g. RAM or ROM, storage means, e.g. floppy disk or hard disk units and input/output units.

A basic idea of the present invention is to use the bio-impedance sensing means, i.e. the field sensors of the apparatus, which originally are adapted to measure a resulting secondary magnetic field in order to obtain bio-impedance values, not only for measuring bio-impedance, but also for measuring a number of reference signals, i.e. for determining the position and/or motion of the user's body. In other words the range of application of those sensing means is increased. That is, the apparatus for measuring bio-impedance is used at the same time as an apparatus for determining the position and/or motion of the user's body. As a result, the number of components necessary to operate the position and/or motion detection is drastically reduced.

According to the invention the position and/or the motion of the user's body is determined by analyzing the position of the two or more reference signal generators, which are attached to the user's body. This is achieved according to another basic idea of the invention by analyzing the results of the bio-impedance measurements. For example the bio-impedance measurements result in a tomographic picture, from which the position and/or motion of each reference signal generator are determined. No additional steps, e.g. use of a triangulation system or the like, has to be applied. The position and/or motion detection is carried out simply by analyzing the bio-impedance measuring results. The effort for determining the position and/or the motion of a user's body is thus reduced noticeably.

These and other aspects of the invention will be further elaborated on the basis of the following embodiments, which are defined in the dependent claims.

According to the invention a number of reference signal generators are proposed, which are attached to the user's body at well-defined positions. For carrying out the invention the reference signal must be distinguishable from the objects of interest with respect to the bio-impedance measurement.

This is achieved according to a preferred embodiment of the invention by the use of reference signal generators, which exhibit a shape, which allows the generators to be distinguished from any part of the user's body. If a reference signal generator exhibits a typical shape, e.g. showing a round or elliptical profile, it can be determined as a foreign body or contaminant within the user's body by analyzing the tomographic pictures resulting from the bio-impedance measurement. In other words, the reference signal generators “passively” generate a reference signal due to their presence within the magnetic field. The position and/or motion of the body is determined by analyzing the position and/or motion of the reference signal generators.

Another way of achieving a distinguishable reference signal is using a reference signal generator, which is adapted to actively generate the reference signal. As an example, the invention can be implemented by simply applying a small number of tiny electrical coils to the body of a user, said coils being provided with a battery or the like as a power source and serving as reference signal generators. With a small modification of the analyzing software, which is originally used for analyzing the bio-impedance measuring results, the position and/or movement of the body can be determined in a very reliable way.

According to another embodiment of the invention a distinguishable reference signal is achieved by using a reference signal, which alternates in strength. This is the preferred way and most of the information disclosed in this description relates to an embodiment of the invention using this approach. An advantage of this approach is, that there are no severe requirements on the physical size of the reference signal generators.

To achieve an alternating strength, according to another preferred embodiment of the invention, the reference signal generators are adapted to generate an alternating reference signal, i.e. an alternating magnetic field. Accordingly each reference signal generator preferably comprises a coil, that is driven by a sinusoidal current or the like. The coil, which acts as a dipole during bio-impedance measurement, is connected to a built-in energy source, e.g. a small battery or the like. In other words, each reference signal generator is preferably designed to work autonomously as a stand-alone device. To distinguish a number of reference signal generators from each other it is possible to use reference signal generators adapted to send reference signals using different frequencies.

According to another preferred embodiment of the invention the reference signal generators are attached in a fixed manner to the user's body, preferably to the user's neck, sternum and/or ilium. In other words each reference signal generator is secured from movement with respect to at least a portion of the user's body. The reference signal generators are preferably attached to the body by means of non-metallic fixing members.

The number of reference signal generators are attached e.g. to the user's neck, sternum, ilium, thorax or nose, depending on the desired arrangement as described below.

According to another preferred embodiment of the invention the position of the reference signal generators at the user's body is selected such, that any translation and rotation of the user's body can be determined unambiguously. This is preferably achieved by a non-symmetrical arrangement of the reference signal generators. In other words, the reference signal generators are placed in a way that spatial orientation and movements of the user can be inferred. In most cases three or four reference signal generators are sufficient to achieve an unambiguous determination of the user's position at any time. Preferably, the reference signal generators are arranged in a manner that they form a tetraeder. If each reference signal generator possesses a dipole, i.e. an aligned marker, such a tetraedric configuration can be reduced to using two reference signal generators, whose dipoles rotate against each other. Using such an arrangement a three-dimensional tracking of the body's position is possible by analyzing the position of the reference signal generators.

According to another preferred embodiment of the invention the analyzing means are adapted to amend and/or adjust the bio-impedance values depending on the results of the position detection and/or motion detection. Preferably the results of the position detection and/or motion detection are used by the analyzing means to subtract motion artifacts and/or for calibration purposes, i.e. to calibrate the measuring apparatus.

The apparatus according to the invention is preferably used for determining the position of the user's body in the form of a discrete, i.e. single measurement. According to a preferred embodiment of the invention at least one of the reference signal generators operates before and/or after the alternating magnetic field is induced in the user's body. In other words, the measuring procedure is preferably as follows: First, the number of reference signal generators are put into operation, i.e. each reference signal generator is switched on. The reference signal generators are preferably adapted to generate reference signals with high intensity or amplitude. If the reference signal generators are put into operation, the position detection is carried out, i.e. the sensing means measures the reference signal. In a next step the reference signal generators are switched off. Subsequently, the actual bio-impedance measurement is carried out, i.e. scanning the lung of the user or the like.

In this case a very simple approach for generating the reference signal is that the same frequency is used as when an alternating magnetic field is subsequently induced in the user's body by the generating means. Because the two signals or fields are not “sent” at the same time, no differentiation is necessary. If the reference signal generators are sending the reference signal, which preferably is an alternating magnetic field to be sensed by the bio-impedance sensing means, the resulting “bio-impedance picture” shows the signals of the reference signal generators only. If the bio-impedance measuring is taking place, no reference signal generator signals are sensed and displayed. Of course, also a different frequency may be used by the reference signal generators.

The measuring of the position of the reference signal generators can be carried out as a one-time measurement, e.g. for the purpose of a one-time determination of the position and/or orientation of the patient. Alternatively, the position detection can be carried out several times, preferably before and after the alternating magnetic field is induced in the user's body, i.e. before and after the bio-impedance measurement. If the position detection is carried out at regular intervals and/or during a longer period of time, long-term scans with position tracking can be performed, e.g. overnight or over several days and/or nights. If the activation and deactivation of the reference signal generators occurs within a sufficient short time interval, e.g. once per second or once per minute, depending on the kind of bio-impedance measurement, a quasi-continuous position detection is possible, which can be used also for motion analysis of the user's body. Additionally or alternatively, a position detection can be performed each time a number of certain predetermined parameter are met, e.g. a certain time period has expired or a certain time of day is reached or the blood pressure or any other parameter of the user meets a certain predetermined or dynamically determined threshold or critical value.

According to another preferred embodiment of the invention the apparatus is used for determining the motion of the user's body in the form of a continuous measurement. That is, at least one of the signal generators operates at the same time when the alternating magnetic field is induced in the user's body. In other words the measuring procedure is preferably as follows: First, the number of reference signal generators are put into operation. The reference signal generators are preferably adapted to generate reference signals using another frequency than for the subsequent inducing of an alternating magnetic field in the user's body by the generating means. Alternatively, another distinguishing feature is implemented, e.g. the use of typically shaped reference signal generators. In a next step, bio-impedance measurement is carried out, i.e. scanning the lung of the user or the like. In other words the bio-impedance measurement, i.e. the measuring of a secondary magnetic field to obtain bio-impedance values, and the measuring of the reference signal, is carried out in parallel by the same sensing means. The reference signal generators are deactivated if the bio-impedance measurement is finished. This means, that the position of the reference signal generators and thus the position and the motion of the user's body is determined throughout the whole bio-impedance measurement.

These and other aspects of the invention will be described in detail hereinafter, by way of example, with reference to the following embodiments and the accompanying drawings; in which:

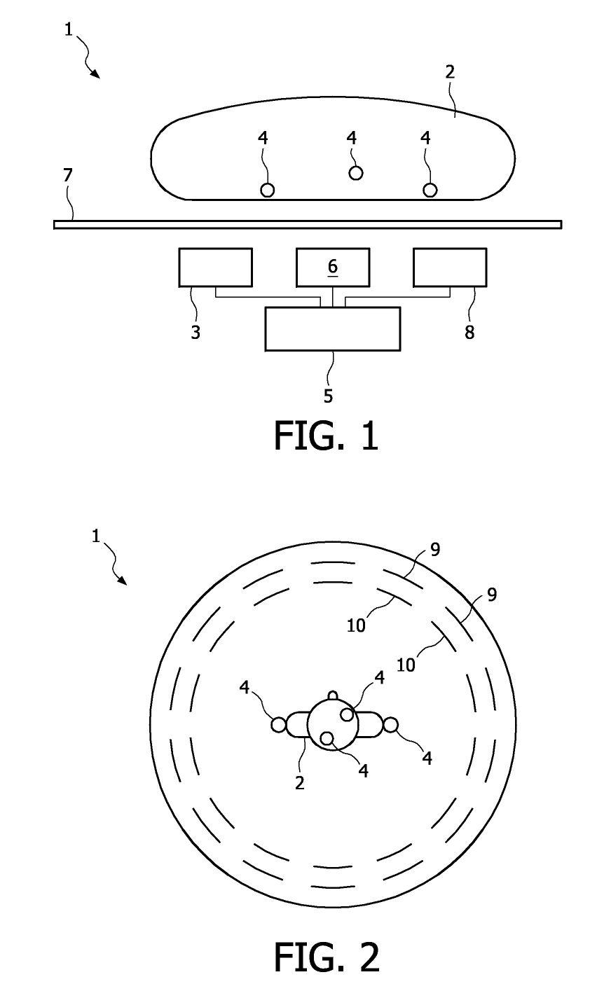

FIG. 1 is a schematic view of an apparatus for inductively measuring the bio-impedance of a user's body according to the invention,

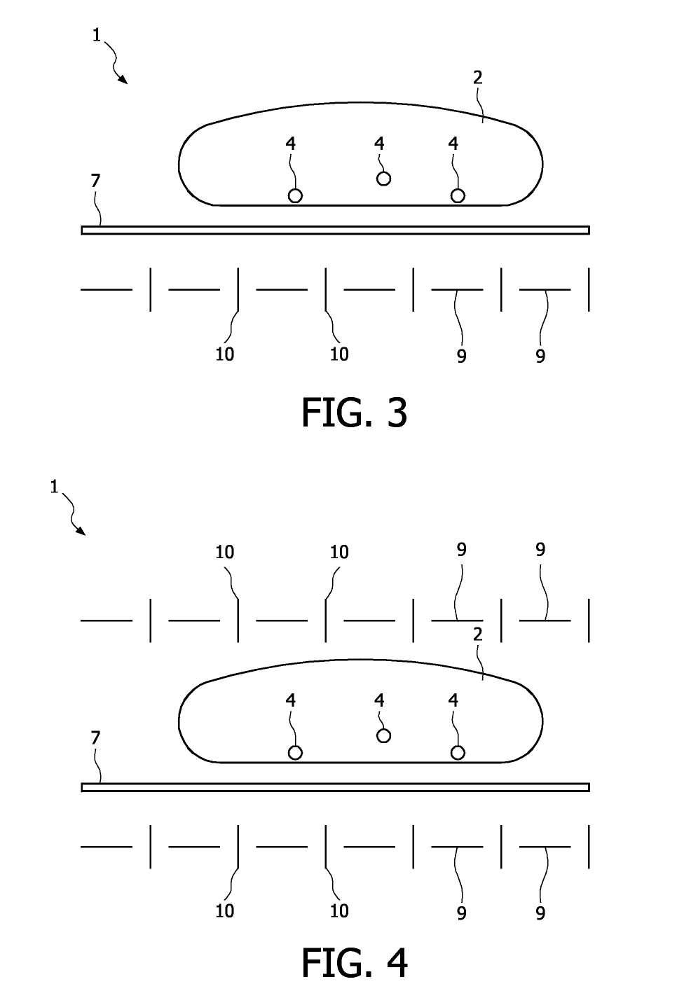

FIG. 3 is a schematic view of an apparatus according to the invention with excitation and measurement coils embedded in the base of a bed,

FIG. 4 is a schematic view of an apparatus according to the invention with excitation and measurement coils also above the patient,

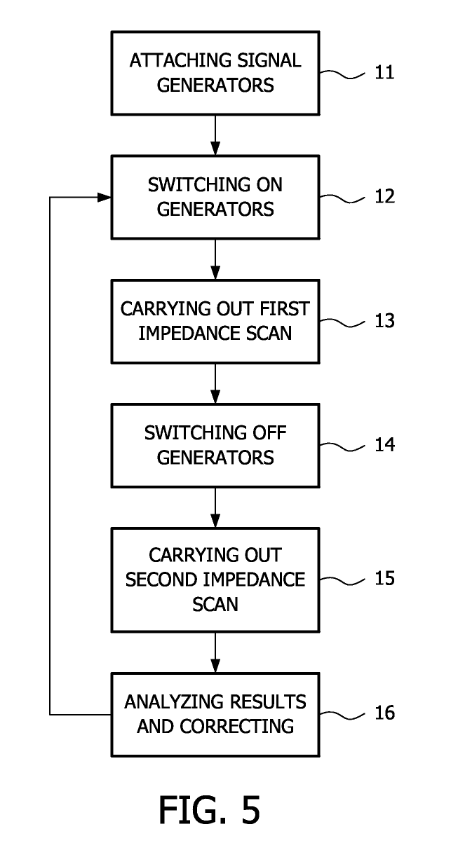

FIG. 5 is a flow chart of a method according to a first embodiment, and

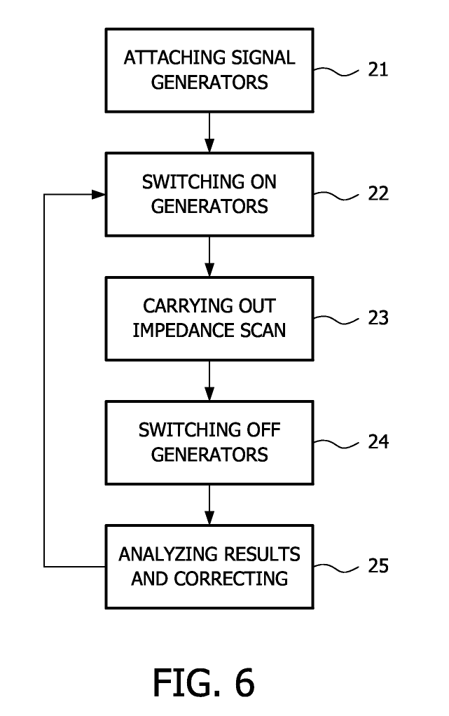

FIG. 6 is a flow chart of a method according to a second embodiment of the invention.

FIG. 1 illustrates a simplified schematic view of an apparatus 1 for inductively measuring the bio-impedance of a user's body 2. The apparatus 1 is adapted to generate tomographic pictures of the user's body 2. In other words a three-dimensional picture of the body 2 is produced.

The apparatus 1 comprises a generating unit 3 which is adapted to induce an alternating magnetic field in the user's body 2. The generating unit 3 comprises a number of excitation or generating coils (not shown in detail) adapted to generate the magnetic field. These generating coils generate an alternating magnetic field. Each generating coil is driven by a sinusoidal current provided by an appropriate circuitry (not shown in detail). The operation is as follows: The generating coils are switched on one by one. If one generating coil is switched on, all the measurement coils (pick-up coils, see below) measure.

The apparatus 1 further comprises four reference signal generators 4 attached to the user's body 2. Each reference signal generator 4 is adapted to actively generate a distinguishable reference signal. For this purpose, each reference signal generator 4 comprises an electrical coil (not shown) adapted to generate an alternating magnetic field. Each coil is driven by a sinusoidal current provided by appropriate circuitry (not shown in detail) The frequency of the reference signal generators can be the same as the measurement coils. Alternatively, the frequency of the reference signal generators is slightly different from the frequency of the measuring coils. The applied frequency depends on the measurement strategy. Furthermore each reference signal generator 4 comprises a battery or the like (not shown) as a power source for the coil. The circuitry comprises an activating/deactivating element (not shown), e.g. an ON/OFF switch. Preferably, the ON/OFF switches of all reference signal generators 4 attached to the user 2 are controllable by means of an external central control unit 5.

The reference signal generators 4 are fixedly attached to the user's body 2. Three of the four reference signal generators 4 are attached to the user's neck, sternum and ilium. The fourth reference signal generator is also attached to the user's torso (not shown). The four reference signal generators 4 are arranged in a non-symmetrical way so that they form a tetraeder. The reference signal generators 4 are adapted to actively generate a reference signal. In other words, each active reference signal generator can be considered to be a dipole. The resulting magnetic field exhibits a certain direction, i.e. two active reference signal generators are enough to determine the position of the body from the direction of the magnetic field.

The reference signal generators 4 can e.g. be worn under clothing as well as in clothing and allow free movement of the user's body 2.

The apparatus 1 further comprises a sensing unit 6 adapted to carry out a magnetic induction measurement. The sensing unit 6 is adapted to measure a secondary magnetic field to obtain bio-impedance values. Furthermore, the sensing unit 6 is adapted to measure a number of reference signals. In other words the sensing unit 6 does not only sense the resulting secondary magnetic field in order to obtain bio-impedance values, but also senses the alternating magnetic field which is generated as a reference signal by means of the number of reference signal generators 4.

The sensing unit 6 comprises a number of pick-up coils (not shown) attached to the user's bed 7. The pick-up coils are arranged in the form of a fixed coil array in a way that position and/or motion of the user's body 2 can be determined irrespective of the user's position on the bed 7. In other words, the user may e.g. assume typical sleeping positions, rotate, move etc. There are different ways how the pick up or measuring coils can be arranged. One possibility is that they are arranged underneath the bed as a planar array. There are also other arrangements possible, e.g. by measuring the magnetic gradient in different directions. The coils can be arranged in a position axial to the patient, as illustrated in FIG. 2, which shows a bio-impedance tomograph. The patient is located in the center of a circular arrangement of excitation coils 9 and pick-up coils 10. This ring of coils may move along the user's axis to cover the full height of the user. The user wears reference signal generators 4 as described above. In another embodiment, as illustrated in FIG. 3, the pick-up coils are positioned on one side of the bed 7 only. In this embodiment, excitation and pick-up coils 9, 10 are embedded in the base of the bed 7 in a planar array. Again, the user wears reference signal generators 4 as described above. In yet another embodiment of the invention the coils 9, 10 are arranged above and beneath the user, as illustrated in FIG. 4. This embodiment is a variation of the bed arrangement shown in FIG. 3, which is complemented by excitation and pick-up coils 9, 10 also above the user. Which of the three embodiments is most useful depends on the individual measuring situation. However, a preferred embodiment would be the use of a coil arrangement below the bed 7, since any standard bed can be equipped with such a system. In an alternative embodiment an arrangement is used, wherein the coils of the generating unit 3 are used as pick-up coils.

The apparatus 1 further comprises an analyzing unit 8. The analyzing unit 8 is adapted to generate a three-dimensional picture of the user's body 2 based on the bio-impedance measurement results. Furthermore the analyzing unit 8 is adapted to determine the position and/or the motion of the user's body 2 based on the measured reference signals. In other words the analyzing unit 8 is adapted for reconstruction (calculation) of the position of the reference signal generators 4 in a three-dimensional space to derive information on the orientation, position or motion of the user's body 2 to which the reference signal generators 4 are attached, based on the resulting tomographic bio-impedance picture. Different known methods can be used to easily detect the reference signal generators 4 with a regular bio-impedance scan, e.g the Tikhonov regularization. As a result the motion of the user's organs, e.g. lung etc., can be determined, based on the position or motion of the user itself. As a result, the bio-impedance measuring results can be interpreted with regard to those position and/or motion data. The analyzing unit 8 also determines three-dimensional or two-dimensional conductivity distributions by reconstruction of the pictures. Additionally, the analyzing unit 8 is adapted to amend and/or adjust the bio-impedance values depending on the results of the position detection and/or motion detection.

The analyzing means 8 preferably comprises a computer (not shown). The computer is adapted to execute computer instructions in the form of a software program. Said computer instructions are adapted in a way that by analyzing the bio-impedance measuring results, the position and/or movement of the user's body 2 is determined, when the computer program is executed in a computer. Additionally, the computer instructions are adapted in a way that the bio-impedance values are amended and/or adjusted depending on the results of the position detection and/or motion detection, e.g. by subtraction of motion artifacts and/or calibration, when the computer program is executed in a computer.

For the purpose of controlling the operation of the apparatus 1, the analyzing unit 8, the sensing unit 6 and the generating unit are connected to the central control unit 5 for switching the reference signal generators 4 on and off. The central control unit 5 preferably comprises a computer (not shown). The computer is adapted to execute computer instructions in the form of a software program, said computer instructions are adapted in a way that all the operational functions of the apparatus 1 are controlled in a predetermined manner, when the computer program is executed in a computer.

In FIG. 5 a first embodiment of a method for inductively measuring the bio-impedance of a user's body 2 according to the present invention is illustrated, wherein the position of the user's body 2 is determined. For this purpose, a number of reference signal generators 4 are attached to the user's body 2 in a first step 11. In the next step 12, the reference signal generators 4 are switched on and the position detection is carried out by means of a first impedance scan for the reference signal generators in step 13, i.e. the sensing unit 6 measures the alternating magnetic field generated by the reference signal generators 4. After the reference signal generators 4 have been switched off in the next step 14, the actual bio-impedance scan for the user's body 2 is carried out in step 15. For this purpose, an alternating magnetic field is induced in the user's body 2 by the generating unit 3 and a secondary magnetic field is measured by the sensing unit 6 in order to obtain the bio-impedance measuring values. Finally, the analyzing unit 8 determines the position of the user's body 2 based on the measured position signals and amends and/or adjusts the results of the bio-impedance measurement in step 16. The reference signal generators 4 are activated continuously each time before a bio-impedance measurement is started. Thus, the results of the position detection can be used to calibrate the bio-impedance measurements before scanning, which results in an enhanced bio-impedance signal quality.

In FIG. 6 a second embodiment of a method for inductively measuring the bio-impedance of a user's body 2 according to the present invention is illustrated, wherein the motion of the user's body 2 is determined. Again a number of reference signal generators 4 are attached to the user's body 2 in a first step 21. In the next step 22 reference signal generators 4 are switched on. In a next step 23, the bio-impedance measurement is carried out. For this purpose, an alternating magnetic field is induced in the user's body 2 by the generating unit 3 and a resulting secondary magnetic field is measured by the sensing unit 6 in order to obtain the bio-impedance measuring values. If the bio-impedance measurement is finished, the reference signal generators 4 are switched off in step 24.

During the measuring period the sensing unit 6 senses both the resulting secondary magnetic field caused by the magnetic field induced in the user's body 2 and the alternating magnetic field caused by the reference signal generators 4. The analyzing unit 6 determines the position and the motion of the user's body 2 based on the measured position signals. In order to distinguish the signals within the resulting three-dimensional bio-impedance picture, the reference signal generators 4 use another frequency for generating the alternating magnetic field than the generating unit 6 uses for inducing the magnetic field in the user's body 2. Again the analyzing unit 8 generates a three-dimensional bio-impedance picture and amends and/or adjusts the results of the bio-impedance measurement in a final step 25. The results of the motion detection are used to eliminate motion artifacts from the bio-impedance measuring results.

It will be evident to those skilled in the art that the invention is not limited to the details of the above illustrative embodiments, and that the present invention may be embodied in other specific forms without departing from the spirit or essential attributes thereof. The present embodiments are therefore to be considered in all respects to be illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit may fulfil the functions of several means recited in the claims. Any reference signs in the claims shall not be construed as limiting the claim concerned.

### LIST OF REFERENCE NUMBERS

1 apparatus
2 user's body
3 generating unit
4 reference signal generator central control unit
6 sensing unit
7 user's bed
8 analysing unit
9 excitation coil
10 measuring (pick-up) coil
11-25 method steps

1. An apparatus for inductively measuring the bio-impedance of a user's body, comprising:
generating means, which are adapted to induce an alternating magnetic field in the user's body,
a number of reference signal generators attached to the user's body, each reference signal generator being adapted to generate a reference signal,
sensing means which are adapted to measure a secondary magnetic field to obtain bio-impedance values and further being adapted to measure a number of reference signals, and
analyzing means, adapted to determine the position and/or the motion of the user's body based on the measured reference signals.

2. The apparatus as claimed in claim 1, the reference signal generators exhibit a shape, which allows the reference signal generators to be distinguished from any part of the user's body.

3. The apparatus (1) as claimed in claim 1, the reference signal generators are adapted to actively generate the reference signal.

4. The apparatus as claimed in claim 1, the reference signal generators are adapted to generate an alternating reference signal.

5. The apparatus as claimed in claim 1, the reference signal generators are attached in a fixed manner to the user's body.

6. The apparatus as claimed in claim 1, the number of reference signal generators is chosen such that any translation and rotation of the user's body can be determined unambiguously.

7. The apparatus as claimed in claim 1, the analyzing means are adapted to amend and/or adjust the bio-impedance values depending on the results of the position detection and/or motion detection.

8. A method for inductively measuring the bio-impedance of a user's body, said method comprising the steps of:
attaching a number of reference signal generators to the user's body,
operating the reference signal generators such that a number of reference signals is actively generated,
inducing an alternating magnetic field in the user's body by generating means
measuring a secondary magnetic field by sensing means in order to obtain bio-impedance values,
measuring a number of reference signals by said sensing means, and
determining the position and/or the motion of the user's body based on the measured reference signals by analyzing means 8.

9. The method as claimed in claim 8, at least one of the reference signal generators operates before and/or after the alternating magnetic field is induced in the user's body.

10. The method as claimed in claim 8, at least one of the signal generators operates at the same time when the alternating magnetic field is induced in the user's body.

11. The method as claimed in claim 8, the further step of amending and/or adjusting the bio-impedance values depending on the results of the position detection and/or motion detection.

12. A computer program for use in a method for inductively measuring the bio-impedance of a user's body, said method comprising the steps of:
attaching a number of reference signal generators to the user's body,
operating the reference signal generators such that a number of reference signals is actively generated,
inducing an alternating magnetic field in the user's body by generating means,
measuring a secondary magnetic field by sensing means in order to obtain bio-impedance values,
measuring a number of reference signals by said sensing means, said computer program comprising computer instructions to determine the position and/or the motion of the user's body based on the measured reference signals, when the computer program is executed in a computer.

13. An apparatus for inductively measuring the bio-impedance of a patient comprising:
a generating unit that induces an alternating magnetic field in the patient;
a plurality of reference signal generators disposed proximate to the patient;
a sensing unit that measures a secondary magnetic field to obtain bio-impedance values and measures one or more reference signals from the reference signal generators;
an analyzing unit that determines the position or motion of the patient based on the measured reference signals.

14. The apparatus of claim 13, wherein the reference signal generators exhibit a shape that permits the reference signal generators to be distinguished from any portion of the patient.

15. The apparatus of claim 13, wherein the analyzing unit adjusts the bio-impedance values based on the determined position or motion.
